(19) United States
(12) Patent Application Publication
(10) Pub. No.: US 2004/0154109 A1
(43) Pub. Date: Aug. 12, 2004

(54) Composition for dyeing keratin fibers, comprising at least one dialdehyde heterocyclic compound and at least one nitrogen compound

(76) Inventor: Gregory Plos, Paris (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 10/724,083

(22) Filed: Dec. 1, 2003

Related U.S. Application Data
(60) Provisional application No. 60/432,981, filed on Dec. 13, 2002.
(60) Provisional application No. 60/439,981, filed on Jan. 14, 2003.

(30) Foreign Application Priority Data
Nov. 29, 2002 (FR) 02 15058

(51) Int. Cl. A61K 7/13

(52) U.S. Cl. 8/405

(57) ABSTRACT

The invention relates to a dye composition for dyeing keratin fibers, comprising at least one heterocyclic compound containing at least two aldehyde functional groups and at least one nitrogen compound. The invention also relates to the use of this composition for dyeing keratin fibers, and to a multi-compartment dyeing device using this composition.

This application claims benefit of U.S. Provisional Application No. 60/432,981, filed Dec. 13, 2002.

The present invention relates to a dye composition for dyeing keratin fibers, for example, human keratin fibers such as the hair, comprising at least one heterocyclic compound containing at least two aldehyde functions in α, β or γ positions of the heterocyclic compound and at least one nitrogen compound.

Two main modes of dyeing exist in the field of dyeing keratin fibers—for example, human keratin fibers such as the hair—these modes each having their advantages and drawbacks:

direct dyeing or semi-permanent dyeing comprises introducing the color via a colored molecule, which may be adsorbed onto the surface of the keratin fibers and/or may penetrate by diffusion into the surface layers of these fibers. The leave-in times may generally be fairly short and the mild dyeing conditions may preserve the integrity of the keratin fibers, but the colorations obtained by this mode of dyeing also may show poor wash fastness and may fade out after shampooing only 4 or 5 times. Furthermore, the ranges of shades obtained may be generally small;

oxidation dyeing or permanent dyeing uses the oxidative condensation of colorless or weakly colored molecules, known as oxidation bases, such as ortho- or para-phenylenediamines, ortho- or para-aminophenols or heterocyclic compounds in the presence of an oxidizing agent. This reaction may lead to the formation of colored polymer compounds in the keratin fibers. The main advantages of oxidation dyeing may lie in the longevity of the colorations obtained, such as in the fastness to washing and to external agents such as light, bad weather, permanent waving, perspiration and rubbing, and also in the production of a wide range of shades. However, the chemical dyeing conditions, such as the pH and the presence of an oxidizing medium, may result in degradation of the keratin fibers. Moreover, this mode of dyeing may require relatively long leave-in times.

Systems for dyeing keratin fibers using an aldehyde compound in the presence of an amine without using oxidizing agents that degrade the hair already exist. Patent FR 2 787 705 proposes compositions for dyeing keratin fibers, comprising an aliphatic cationic amine and a compound chosen from an aldehyde, a ketone, a quinone and a diiminoisoindoline or 3-aminoisoindolone derivative, and patent FR 2 787 706 proposes compositions for dyeing keratin fibers, comprising a heterocyclic cationic amine and a compound chosen from an aldehyde, a ketone, a quinone and a diiminoisoindoline or 3-aminoisoindolone derivative.

U.S. Pat. Nos. 3 871 818 and 3 904 357 describe a process for dyeing the hair with dialdehydes in the presence of at least one nitrogen compound. These documents disclose the combination of a xanthenedicarboxaldehyde on the one hand, and of a piperidinedicarboxaldehyde on the other hand, with a compound containing a nitrogen atom.

However, the colorations obtained using these dye compositions may not always be strong enough, aesthetic enough, chromatic enough or sufficiently fast with respect to the various attacking factors to which the hair may be subjected.

Disclosed herein, therefore, are novel dye compositions for dyeing keratin fibers that do not have at least one of the drawbacks of those of the prior art. For example, disclosed herein is a novel dyeing system that presents at least one of the advantages of resistance, such as to repeated washing, and can also show respect for the hair fiber.

Thus, in one embodiment, disclosed herein is a composition for dyeing keratin fibers, comprising, in a suitable dyeing medium:

a) at least one polyaldehyde heterocyclic compound of formula (I) comprising at least two aldehyde functional groups in α, β or γ positions on the at least one polyaldehyde heterocyclic compound, or a corresponding addition salt thereof:

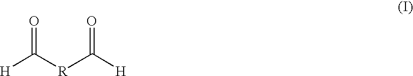

wherein R is chosen from a 5- to 30-membered aromatic or non-aromatic, fused or non-fused, unsaturated divalent monoheterocyclic or polyheterocyclic group comprising at least one hetero atom chosen from nitrogen, sulphur, oxygen and phosphorus,

these heterocyclic groups optionally being substituted with at least one group chosen from halo, C₁-C₄ alkyl, hydroxyl, C₁-C₄ alkoxy, C₁-C₄ alkylthio, amino, C₁-C₄ monoalkylamino, di(C₁-C₄)alkylamino, C₁-C₄ alkylcarbonyl, hydrogenocarbonyl, C₁-C₄ alkoxycarbonyl, nitro, sulphonato, ammonio, tri(C₁-C₄)alkylammonio, imidazolio, pyridinio and benzothiazolio groups; and

b) at least one nitrogen compound of formula (II) or a corresponding addition salt thereof:

NR₁R₂R₃ (II)

wherein R₁, R₂ and R₃, independently of each other, are chosen from:

a hydrogen atom;

a hydroxyl group;

a C₁-C₄ alkoxy group;

a branched or unbranched C₁₋₃₀ aliphatic chain, which may optionally comprise at least one bond chosen from double and triple bonds, optionally substituted with at least one group chosen from hydroxyl, amino and halo groups;

a fused or non-fused monoaromatic or polyaromatic group comprising from 6 to 50 carbon atoms; and

a 5- to 30-membered aromatic or non-aromatic, fused or non-fused, monoheterocyclic or polyheterocyclic group comprising at least one hetero atom chosen from nitrogen, sulphur, oxygen and phosphorus;

these heterocyclic groups optionally being substituted with at least one group chosen from halo, C₁-C₄ alkyl, hydroxyl, C₁-C₄ alkoxy, C₁-C₄ alkylthio, amino, C₁-C₄ monoalkylamino, di(C₁-C₄)alkylamino, C₁-C₄ alkylcarbonyl, hydrogenocarbonyl, carboxyl, nitro and sulphonato groups.

The compositions disclosed herein may make it possible to obtain fast shades, such as shampoo-fast shades. These compositions may also have the advantage of limiting the degradation of hair fibers since the keratin fiber is not in contact with strong oxidizing agents such as hydrogen peroxide usually used for oxidation dyeing.

Also disclosed herein are a process for dyeing keratin fibers, for example human keratin fibers such as the hair, using this composition, and also a dyeing device for performing this process.

As used herein, the term “at least two aldehyde functional groups in α, β or γ positions on the at least one polyaldehyde heterocyclic compound” means that the compound of formula (I) comprises at least two aldehyde functional groups located on the mono- or poly-heterocyclic group R, wherein the at least two aldehyde functional group(s) are located on neighboring ring atoms of R (i.e., in the a position with respect to each other) or on ring atoms of R separated by one or two ring atoms (i.e., such that the aldehyde functional groups are β or γ to each other). The term “ring atoms of R” as used herein means either carbon atoms or hetero atoms.

As used herein, the term “fused” means at least two rings joined together containing at least two atoms in common.

As used herein, the term “alkyl radical” (“alk”) means a linear or branched alkyl radical, for example methyl, ethyl, n-propyl, isopropyl or butyl. As used herein, an alkoxy radical is a radical alk-O—, an alkylcarbonyl radical is a radical alk-CO—, an alkoxycarbonyl radical is a radical alk-O—CO—, an alkylthio radical is a radical alk-S—, a monoalkylamino or dialkylamino radical is a radical (alk)ₙN— with n=1 or 2, in each of these definitions the alkyl radical having the definition given above.

As used herein, a sulphonato radical is an —SO₃⁻radical, and a trialkylammonio radical is a radical (alk)₃N⁺— with the alkyl radical having the definition given above.

As used herein, the imidazolio, pyridinio and benzothiazolio radicals are cationic radicals corresponding to the imidazolium, pyridinium and benzothiazolium cations.

As used herein, a halo group denotes a halogen atom chosen, for example, from chlorine, bromine and iodine.

A fused or non-fused monoaromatic or polyaromatic group containing from 6 to 50 carbon atoms may be, for example, a benzene, naphthalene or anthracene ring system. A 5- to 30-membered aromatic or non-aromatic, fused or non-fused, monoheterocyclic or polyheterocyclic group comprising at least one hetero atom may be, for example, a thiophene, benzofuran, benzothiophene, indole, bispyridine, benzopyran, quinoline, pyrazole, pyridine, pyrrole, furan, imidazole or benzimidazole ring system. The polyheterocyclic group may be fused or substituted with one or more carbocycles.

In one embodiment of the composition disclosed herein, the at least one polyaldehyde heterocyclic compound of formula (I) is a dialdehyde.

In another embodiment of the composition disclosed herein, the at least one polyaldehyde heterocyclic compound of formula (I) is non-fused.

In another embodiment, the at least one polyaldehyde heterocyclic compound of formula (I) is chosen from thiophenedicarboxaldehydes and derivatives thereof, pyridinedicarboxaldehydes and derivatives thereof, pyrroledicarboxaldehydes and derivatives thereof, and furandicarboxaldehydes and derivatives thereof. For example, the at least one polyaldehyde heterocyclic compound of formula (I) may be chosen from 2,3-thiophenedicarboxaldehyde, 2,6-pyridinedicarboxaldehyde, 3,4-dimethyl-2,5-pyrroledicarboxaldehyde, 2,5-thiophenedicarboxaldehyde and 2,5-diformyl-3,4-furandicarboxaldehyde. As a further example, the at least one polyaldehyde heterocyclic compound of formula (I) may be a 5-membered heterocyclic compound, for example 2,3-thiophenedicarboxaldehyde, 3,4-dimethyl-2,5-pyrroledicarboxaldehyde or 2,5-thiophenedicarboxaldehyde.

In one embodiment of the composition disclosed herein, when at least one of the radicals R₁, R₂ or R₃ of formula (II) is chosen from a fused or non-fused, monoaromatic or polyaromatic group comprising from 6 to 50 carbon atoms, the aromatic group is unsubstituted or substituted with at least one group chosen from halo, C₁-C₄ alkyl, hydroxyl, C₁-C₄ alkoxy, C₁-C₄ alkylthio, amino, C₁-C₄ monoalkylamino or di(C₁-C₄)alkylamino, C₁-C₄ alkylcarbonyl, hydrogenocarbonyl, carboxyl, nitro and sulphonato groups.

In one embodiment of the composition disclosed herein, the at least one nitrogen compound of formula (II) is chosen from monoalkanolamines, dialkanolamines, trialkanolamines, alkylalkanolamines, dialkylalkanolamines and alkyldialkanolamines.

The at least one nitrogen compound of formula (II) may be chosen from C₁-C₃ monoalkanolamines, di(C₁-C₃)alkanolamines, tri(C₁-C₃)alkanolamines, (C₁-C₄ alkyl)(C₁-C₃ alkanol)amines, (di(C₁-C₄)alkyl)(C₁-C₃ alkanol)amines and (C₁-C₄ alkyl) (di(C₁-C₃)alkanol)amines. For example, among the nitrogen compounds of formula (II) that may be used, mention may be made of monoethanolamine, triethanolamine, 2-methyl-1-propanol and monoisopropanolamine.

In another embodiment, the at least one nitrogen compound of formula (II) may be chosen from aromatic amines and diamines. Examples that may be mentioned include para-phenylenediamines, bis(phenyl)alkylenediamines, para-aminophenols, ortho-aminophenols, meta-phenylenediamines, meta-aminophenols, and heterocyclic bases and couplers containing at least one amine function.

Among the para-phenylenediamines that may be used herein, mention may be made of para-phenylenediamine, para-toluenediamine, 2-chloro-para-phenylenediamine, 2,3-dimethyl- para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-N,N-bis(β-hydroxyethyl)amino-2-methylaniline, 4-N,N-bis(β-hydroxyethyl)amino-2-chloroaniline, 2-β-hydroxyethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N-ethyl-N-(β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-β-hydroxyethyloxy-para-phenylenediamine, 2-β-acetylaminoethyloxy-para-phenylenediamine, N-(β-methoxyethyl)-para-phenylenediamine, 4-aminophenylpyrrolidine, 2-thienyl-para-phenylenediamine, 2-β-hydroxyethylamino-5-aminotoluene and 3-hydroxy-1-(4′-aminophenyl)pyrrolidine, and the acid-addition salts thereof.

For example, the para-phenylenediamines may be chosen from para-phenylenediamine, para-toluenediamine, 2-isopropyl-para-phenylenediamine, 2-β-hydroxyethyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine, and 2-β-acetylaminoethyloxy-para-phenylenediamine, and the acid-addition salts thereof.

In one embodiment, the at least one nitrogen compound of formula (II) is aqueous ammonia.

The concentrations of the at least one polyaldehyde heterocyclic compound of formula (I) and of the at least one nitrogen compound of formula (II) in the composition may vary widely depending on the desired shade. The at least one polyaldehyde heterocyclic compound of formula (I) may be present in an amount ranging from 0.01% to 30% by weight, such as from 0.05% to 20% by weight, relative to the total weight of the dye composition, and the at least one nitrogen compound of formula (II) may be present in an amount ranging from 0.01% to 30% by weight, such as from 0.05% to 20% by weight, relative to the total weight of the dye composition.

The dye composition disclosed herein may also contain at least one direct dye that may be chosen from, for example, nitrobenzene dyes, azo direct dyes and methine direct dyes. These direct dyes may be of nonionic, anionic or cationic nature.

The composition disclosed herein may also comprise at least one oxidation base and/or at least one coupler conventionally used in oxidation dyeing, other than those possibly acting as the at least one nitrogen compound of formula (II). By way of example, these oxidation bases may be chosen from para-diphenols and heterocyclic bases containing no amine function. The couplers may be chosen from meta-diphenols and heterocyclic couplers containing no amine function.

The medium suitable for dyeing, also known as the dye support, generally comprises water or a mixture of water and of at least one organic solvent to dissolve the compounds that would not be sufficiently water-soluble. Examples of organic solvents that may be mentioned include C₁-C₄ lower alkanols, such as ethanol and isopropanol; polyols and polyol ethers, for instance 2-butoxyethanol, propylene glycol, propylene glycol monomethyl ether and diethylene glycol monomethyl ether and monoethyl ether, and also aromatic alcohols, for instance benzyl alcohol or phenoxyethanol, and mixtures thereof.

The solvents may be present in amounts ranging from 1% to 40% by weight approximately, such as from 5% to 30% by weight, relative to the total weight of the dye composition.

The dye composition disclosed herein may also contain at least one of various adjuvants conventionally used in hair dye compositions, such as anionic, cationic, nonionic, amphoteric or zwitterionic surfactants or mixtures thereof, anionic, cationic, nonionic, amphoteric or zwitterionic polymers or mixtures thereof, mineral or organic thickeners, such as anionic, cationic, nonionic and amphoteric polymeric associative thickeners, antioxidants, penetrating agents, sequestrants, fragrances, buffers, dispersants, conditioners, for example volatile or non-volatile, modified or unmodified silicones, film-forming agents, ceramides, preserving agents and opacifiers.

The above adjuvants may individually be present in an amount for each ranging from 0.01% to 20% by weight relative to the weight of the composition.

Needless to say, a person skilled in the art will take care to select this or these optional additional compound(s) such that the advantageous properties intrinsically associated with the dye composition in accordance with the invention are not, or are not substantially, adversely affected by the envisaged addition(s).

The pH of the dye composition in accordance with the invention may range from 4 to 11, such as from 5 to 10. It may be adjusted to the desired value using acidifying or basifying agents usually used in the dyeing of keratin fibers, or alternatively using standard buffer systems.

Among the acidifying agents that may be mentioned, for example, are mineral or organic acids, for instance hydrochloric acid, orthophosphoric acid, sulphuric acid, carboxylic acids, for instance acetic acid, tartaric acid, citric acid or lactic acid, and sulphonic acids.

The nitrogen compound(s) of formula (II) act(s) as basifying agent(s). However, the pH may be adjusted by adding other basifying agents, such as alkaline carbonates and sodium or potassium hydroxide.

The dye composition according to the invention may be in various forms, such as in the form of liquids, creams or gels, or in any other form that is suitable for dyeing keratin fibers, for example, human hair.

The process for dyeing at least one keratin fiber as disclosed herein comprises applying to at least one keratin fiber for a leave-in time that is sufficient to obtain the desired coloration a composition (A) comprising, in a cosmetic medium that is suitable for dyeing the hair, at least one polyaldehyde heterocyclic compound of formula (I) and a composition (B) comprising, in a cosmetic medium that is suitable for dyeing the hair, at least one nitrogen compound of formula (II).

In one embodiment, compositions (A) and (B) are mixed together just before use and the mixture thus obtained is applied to the keratin fibers for a leave-in time that is sufficient to obtain the desired coloration.

According to one variant, the process for dyeing keratin fibers comprises applying compositions (A) and (B) successively to the keratin fibers for a leave-in time that is sufficient to obtain the desired coloration. These compositions may be applied in any order.

According to another embodiment, the keratin fibers are rinsed between the application of the two compositions.

The leave-in time for each of the compositions (A) or (B) or for their mixture may range from 5 minutes to 1 hour, such as from 5 minutes to 30 minutes.

The temperature at which the various compositions are applied may range from room temperature to 80° C., such as from room temperature to 60° C.

The multi-compartment device of the invention comprises at least one first compartment comprising a composition comprising at least one dialdehyde heterocyclic compound of formula (I) and at least one second compartment comprising a composition comprising at least one nitrogen compound of formula (II). This device may be equipped with a means for applying the desired mixture to the hair, such as the devices described in patent FR-2 586 913. By means of this device, it is possible to dye keratin fibers using a process as described above.

Disclosed herein is thus also the use of a composition comprising at least one polyaldehyde heterocyclic compound of formula (I) and at least one nitrogen compound of formula (II) to dye keratin fibers.

The examples that follow serve to illustrate the invention without, however, being limiting in nature.

### EXAMPLES

### Example 1

A dye composition was prepared as indicated below:

| Compounds | Composition 1 | |
|---|---|---|
| 2,3-Thiophenedicarboxaldehyde | 6 × 10⁻³ | mol |
| Aqueous ammonia | 0.8 | g |
| Distilled water | qs 100 | g |

This composition was applied to locks of natural or permanent-waved grey hair containing 90% white hairs. After a leave-in time of 30 minutes at 50° C., the locks were shampooed, rinsed and then dried.

The coloration obtained was measured using a Minolta CM2002 spectrocolorimeter (CSE, illuminant D65, angle 10°).

The calorimetric results are given below:

| | L* | a* | b* | Colour |
|---|---|---|---|---|
| Natural coloured lock | 30.50 | 0.85 | 7.75 | brown |
| Permanent-waved coloured lock | 28.25 | 7.10 | 2.85 | brown |

### Example 2

Compositions 2 and 3 below were prepared:

| Compounds | Composition 2 | Composition 3 |
|---|---|---|
| 3,4-Dimethyl-2,5-pyrrole- | 3 × 10⁻³ mol | — |
| dicarboxaldehyde | | |
| 2,5-Thiophenedicarboxaldehyde | — | 3 × 10⁻³ mol |
| para-Phenylenediamine | 9 × 10⁻³ mol | 9 × 10⁻³ mol |
| NaOH | qs pH 9 | qs pH 9 |
| Distilled water | qs 100 g | qs 100 g |

These compositions were applied to locks of natural grey hair containing 90% white hairs. After a leave-in time of 30 minutes at room temperature, the locks were shampooed, rinsed and then dried.

The colour of the locks was measured using a Minolta CM2002 spectrocolorimeter (CSE, illuminant D65, angle 10°).

The colorimetric results are given below:

| Locks | L* | a* | b* | Colour |
|---|---|---|---|---|
| Composition 2 | 23.60 | 14.05 | 9.45 | coppery |
| Composition 3 | 26.15 | 12.05 | 12.90 | coppery |

What is claimed is:

1. A composition for dyeing keratin fibers, comprising, in a suitable dyeing medium:
a) at least one polyaldehyde heterocyclic compound of formula (I) comprising at least two aldehyde functional groups in α, β or γ positions on the at least one polyaldehyde heterocyclic compound, or a corresponding addition salt thereof: 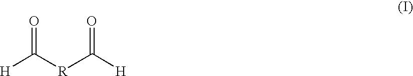
wherein R is chosen from a 5- to 30-membered aromatic or non-aromatic, fused or non-fused, unsaturated divalent monoheterocyclic or polyheterocyclic group comprising at least one hetero atom chosen from nitrogen, sulphur, oxygen and phosphorus,
these heterocyclic groups optionally being substituted with at least one group chosen from halo, C₁-C₄ alkyl, hydroxyl, C₁-C₄ alkoxy, C₁-C₄ alkylthio, amino, C₁-C₄ monoalkylamino or di(C₁-C₄)alkylamino, C₁-C₄ alkylcarbonyl, hydrogenocarbonyl, C₁-C₄ alkoxycarbonyl, nitro, sulphonato, ammonio, tri(C₁-C₄)alkylammonio, imidazolio, pyridinio and benzothiazolio groups;
b) at least one nitrogen compound of formula (II) or a corresponding addition salt thereof:
NR₁R₂R₃ (II)
wherein R₁, R₂ and R₃, independently of each other, are chosen from:
a hydrogen atom;
a hydroxyl group;
a C₁-C₄ alkoxy group;
a branched or unbranched C₁₋₃₀ aliphatic chain, which may comprise at least one bond chosen from double and triple bonds, optionally substituted with at least one group chosen from hydroxyl, amino and halo groups;
a fused or non-fused monoaromatic or polyaromatic group comprising from 6 to 50 carbon atoms; and
a 5- to 30-membered aromatic or non-aromatic, fused or non-fused, monoheterocyclic or polyheterocyclic group comprising at least one hetero atom chosen from nitrogen, sulphur, oxygen and phosphorus;
these heterocyclic groups optionally being substituted with at least one group chosen from halo, C₁-C₄ alkyl, hydroxyl, C₁-C₄ alkoxy, C₁-C₄ alkylthio, amino, C₁-C₄ monoalkylamino or di(C₁-C₄)alkylamino, C₁-C₄ alkylcarbonyl, hydrogenocarbonyl, carboxyl, nitro and sulphonato groups.

2. A composition according to claim 1, wherein the polyaldehyde heterocyclic compound of formula (I) is a dialdehyde.

3. A composition according to claim 1, in which the polyaldehyde heterocyclic compound of formula (I) is non-fused.

4. A composition according to claim 3, wherein the polyaldehyde heterocyclic compound of formula (I) is chosen from thiophenedicarboxaldehydes and derivatives thereof, pyridinedicarboxaldehydes and derivatives thereof, pyrroledicarboxaldehydes and derivatives thereof, and furandicarboxaldehydes and derivatives thereof.

5. A composition according to claim 4, wherein the polyaldehyde heterocyclic compound of formula (I) is chosen from 2,3-thiophenedicarboxaldehyde, 2,6-pyridinedicarboxaldehyde, 3,4-dimethyl-2,5-pyrroledicarboxaldehyde, 2,5-thiophenedicarboxaldehyde and 2,5-diformyl-3,4-furandicarboxaldehyde.

6. A composition according to claim 3, wherein the polyaldehyde heterocyclic compound of formula (I) is a 5-membered heterocyclic compound.

7. A composition according to claim 6, in which the polyaldehyde heterocyclic compound of formula (I) is chosen from 2,3-thiophenedicarboxaldehyde, 3,4-dimethyl-2,5-pyrroledicarboxaldehyde and 2,5-thiophenedicarboxaldehyde.

8. A composition according to claim 1, wherein when at least one of the radicals R₁, R₂ or R₃ of formula (II) is chosen from a fused or non-fused, monoaromatic or polyaromatic group comprising from 6 to 50 carbon atoms, the aromatic group is unsubstituted or substituted with at least one group chosen from halo, C₁-C₄ alkyl, hydroxyl, C₁-C₄ alkoxy, C₁-C₄ alkylthio, amino, C₁-C₄ monoalkylamino or di(C₁-C₄)alkylamino, C₁-C₄ alkylcarbonyl, hydrogenocarbonyl, carboxyl, nitro and sulphonato groups.

9. A composition according to claim 1, wherein the at least one nitrogen compound of formula (II) is chosen from monoalkanolamines, dialkanolamines, trialkanolamines, alkylalkanolamines, dialkylalkanolamines, alkyldialkanolamines, aromatic amines and diamines and aqueous ammonia.

10. A composition according to claim 9, wherein the at least one nitrogen compound of formula (II) is chosen from C₁-C₃ monoalkanolamines, di(C₁-C₃)alkanolamines, tri(C₁-C₃)alkanolamines, (C₁-C₄ alkyl)(C₁-C₃ alkanol)amines, (di(C₁-C₄)alkyl)(C₁-C₃ alkanol)amines and (C₁-C₄ alkyl)(di(C₁-C₃)alkanol)amines.

11. A composition according to claim 10, wherein the at least one nitrogen compound of formula (II) is chosen from monoethanolamine, triethanolamine, 2-methyl-1-propanol and monoisopropanolamine.

12. A composition according to claim 9, wherein the at least one nitrogen compound of formula (II) is chosen from aromatic amines and diamines.

13. A composition according to claim 12, wherein the at least one nitrogen compound of formula (II) is a para-phenylenediamine derivative.

14. A composition according to claim 13, wherein the at least one nitrogen compound of formula (II) is chosen from para-phenylenediamine, para-toluenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-N,N-bis(β-hydroxyethyl)amino-2-methylaniline, 4-N,N-bis(β-hydroxyethyl)amino-2-chloroaniline, 2-β-hydroxyethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N-ethyl-N-(β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-β-hydroxy-ethyloxy-para-phenylenediamine, 2-β-acetylaminoethyloxy-para-phenylenediamine, N-(β-methoxyethyl)-para-phenylenediamine, 4-aminophenylpyrrolidine, 2-thienyl-para-phenylenediamine, 2-β-hydroxyethylamino-5-aminotoluene and 3-hydroxy-1-(4′-aminophenyl)pyrrolidine, and the acid-addition salts thereof.

15. A composition according to claim 14, wherein the at least one nitrogen compound of formula (II) is chosen from para-phenylenediamine, para-toluenediamine, 2-isopropyl-para-phenylenediamine, 2-β-hydroxyethyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine, and 2-β-acetylaminoethyloxy-para-phenylenediamine, and the acid-addition salts thereof.

16. A composition according to claim 9, wherein the at least one nitrogen compound of formula (II) is aqueous ammonia.

17. A composition according to claim 1, wherein the at least one polyaldehyde heterocyclic compound of formula (I) is present in an amount ranging from 0.01% to 30% by weight relative to the total weight of the composition.

18. A composition according to claim 17, wherein the at least one polyaldehyde heterocyclic compound of formula (I) is present in an amount ranging from 0.05% to 20% by weight, relative to the total weight of the composition.

19. A composition according to claim 1, wherein the at least one nitrogen compound of formula (II) is present in an amount ranging from 0.01% to 30% by weight relative to the total weight of the composition.

20. A composition according to claim 19, wherein the at least one nitrogen compound of formula (II) may be present in an amount ranging from 0.05% to 20% by weight, relative to the total weight of the composition.

21. A composition according to claim 1, wherein said composition has a pH ranging from 4 to 11.

22. A composition according to claim 21, wherein said composition has a pH ranging from 5 to 10.

23. A process for dyeing keratin fibers, comprising applying to at least one keratin fiber:
a composition (A) comprising, in a cosmetic medium suitable for dyeing keratin fibers, at least one polyaldehyde heterocyclic compound of formula (I) comprising at least two aldehyde functional groups in α, β or γ positions on the at least one polyaldehyde heterocyclic compound, or a corresponding addition salt thereof: 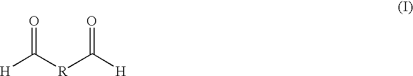
wherein R is chosen from a 5- to 30-membered aromatic or non-aromatic, fused or non-fused, unsaturated divalent monoheterocyclic or polyheterocyclic group comprising at least one hetero atom chosen from nitrogen, sulphur, oxygen and phosphorus,
these heterocyclic groups optionally being substituted with at least one group chosen from halo, C₁-C₄ alkyl, hydroxyl, C₁-C₄ alkoxy, C₁-C₄ alkylthio, amino, C₁-C₄ monoalkylamino or di(C₁-C₄)alkylamino, C₁-C₄ alkylcarbonyl, hydrogenocarbonyl, C₁-C₄ alkoxycarbonyl, nitro, sulphonato, ammonio, tri(C₁-C₄)alkylammonio, imidazolio, pyridinio and benzothiazolio groups; and
a composition (B) comprising, in a cosmetic medium suitable for dyeing the hair, at least one nitrogen compound of formula (II) or a corresponding addition salt thereof:
NR₁R₂R₃ (II)
wherein R₁, R₂ and R₃, independently of each other, are chosen from:
a hydrogen atom;
a hydroxyl group;
a C₁-C₄ alkoxy group;
a branched or unbranched C₁₋₃₀ aliphatic chain, which may comprise at least one bond chosen from double and triple bonds, optionally substituted with at least one group chosen from hydroxyl, amino and halo groups;
a fused or non-fused monoaromatic or polyaromatic group comprising from 6 to 50 carbon atoms; and
a 5- to 30-membered aromatic or non-aromatic, fused or non-fused, monoheterocyclic or polyheterocyclic group comprising at least one hetero atom chosen from nitrogen, sulphur, oxygen and phosphorus;
these heterocyclic groups optionally being substituted with at least one group chosen from halo, C₁-C₄ alkyl, hydroxyl, C₁-C₄ alkoxy, C₁-C₄ alkylthio, amino, C₁-C₄ monoalkylamino or di(C₁-C₄)alkylamino, C₁-C₄ alkylcarbonyl, hydrogenocarbonyl, carboxyl, nitro and sulphonato groups.
wherein said composition (A) and said composition (B) are applied separately or after mixing together, to the keratin fibers for a leave-in time sufficient to obtain a desired coloration.

24. A process according to claim 23, comprising mixing compositions (A) and (B) together just before use and applying the mixture thus obtained to the keratin fibers for a leave-in time sufficient to obtain a desired coloration.

25. A process according to claim 23, comprising successively applying compositions (A) and (B) to the keratin fibers for a leave-in time sufficient to obtain a desired coloration, wherein compositions (A) and (B) may be applied in any order.

26. A process according to claim 23, wherein said leave-in time for each of the compositions (A) or (B) or for their mixture ranges from 5 minutes to 1 hour.

27. A process according to claim 26, wherein said leave-in time ranges from 5 minutes to 30 minutes

28. A process according to claim 25, further comprising rinsing the keratin fibers between the application of composition (A) and the application of composition (B).

29. A multi-compartment device, comprising:
at least one first compartment comprising a composition comprising at least one polyaldehyde heterocyclic compound of formula (I) or an addition salt thereof comprising at least two aldehyde functional groups in α, β or γ positions on the at least one polyaldehyde heterocyclic compound: 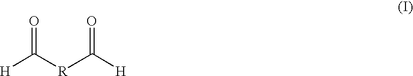
wherein R is chosen from a 5- to 30-membered aromatic or non-aromatic, fused or non-fused, unsaturated divalent monoheterocyclic or polyheterocyclic group comprising at least one hetero atom chosen from nitrogen, sulphur, oxygen and phosphorus,
these heterocyclic groups optionally being substituted with at least one group chosen from halo, C₁-C₄ alkyl, hydroxyl, C₁-C₄ alkoxy, C₁-C₄ alkylthio, amino, C₁-C₄ monoalkylamino, di(C₁-C₄)alkylamino, C₁-C₄ alkylcarbonyl, hydrogenocarbonyl, C₁-C₄ alkoxycarbonyl, nitro, sulphonato, ammonio, tri(C₁-C₄)alkylammonio, imidazolio, pyridinio and benzothiazolio groups; and
at least one second compartment comprising a composition comprising at least one nitrogen compound of formula (II) or a corresponding addition salt thereof:
NR₁R₂R₃ (II)
wherein R₁, R₂ and R₃, independently of each other, are chosen from:
a hydrogen atom;
a hydroxyl group;
a C₁-C₄ alkoxy group;
a branched or unbranched C₁₋₃₀ aliphatic chain, which may comprise at least one bond chosen from double and triple bonds, optionally substituted with at least one group chosen from hydroxyl, amino and halo groups;
a fused or non-fused monoaromatic or polyaromatic group comprising from 6 to 50 carbon atoms; and
a 5- to 30-membered aromatic or non-aromatic, fused or non-fused, monoheterocyclic or polyheterocyclic group comprising at least one hetero atom chosen from nitrogen, sulphur, oxygen and phosphorus;
these heterocyclic groups optionally being substituted with at least one group chosen from halo, C₁-C₄ alkyl, hydroxyl, C₁-C₄ alkoxy, C₁-C₄ alkylthio, amino, C₁-C₄ monoalkylamino or di(C₁-C₄)alkylamino, C₁-C₄ alkylcarbonyl, hydrogenocarbonyl, carboxyl, nitro and sulphonato groups.
